(19) United States
(12) Patent Application Publication
(10) Pub. No.: US 2023/0293868 A1
(43) Pub. Date: Sep. 21, 2023

(54) Disposable Medicinal Wristlet

(71) Applicant: Michavia Izzard, Spartanburg, SC (US)

(72) Inventor: Michavia Izzard, Spartanburg, SC (US)

(21) Appl. No.: 17/697,986

(22) Filed: Mar. 18, 2022

(51) Int. Cl. A61M 35/00; A44C 5/00

(52) U.S. Cl. CPC A61M 35/10; A44C 5/0023

(57) ABSTRACT

A disposable medicinal wristlet includes an elastomeric material formed as a band. A medication within the elastomeric material is operable to be administered in a transdermal manner.

### BACKGROUND

The present disclosure relates to discomfort pertaining to a wearer's wrist.

Discomfort pertaining to a wearer's wrist such as carpal tunnel syndrome affects the median nerve in the wrist. The pressure on this nerve results in pain or numbness in the hand and fingers. Individuals with carpal tunnel syndrome may wear a wrist brace when they have a flare-up or when they are partaking in activities that require prolonged use of the hands. Some may wear the brace during sleep. Disadvantageously, wrist braces may be clunky and in some instances may not be effective.

### SUMMARY

A disposable medicinal wristlet according to one disclosed non-limiting embodiment of the present disclosure includes an elastomeric material formed as a band and a medication within the elastomeric material operable to be administered in a transdermal manner.

The foregoing features and elements may be combined in various combinations without exclusivity, unless expressly indicated otherwise. These features and elements as well as the operation thereof will become more apparent in light of the following description and the accompanying drawings. It should be appreciated that however the following description and drawings are intended to be exemplary in nature and non-limiting.

### BRIEF DESCRIPTION OF THE DRAWINGS

Various features will become apparent to those skilled in the art from the following detailed description of the disclosed non-limiting embodiment. The drawings that accompany the detailed description can be briefly described as follows:

FIG. 1 is a perspective view of a disposable medicinal wristlet according to one disclosed non-limiting embodiment.

FIG. 2 is a perspective view of a disposable medicinal wristlet according to another disclosed non-limiting embodiment.

FIG. 3 is a perspective view of a disposable medicinal wristlet according to another disclosed non-limiting embodiment.

FIG. 4 is a perspective view of a disposable medicinal wristlet according to another disclosed non-limiting embodiment.

### DETAILED DESCRIPTION

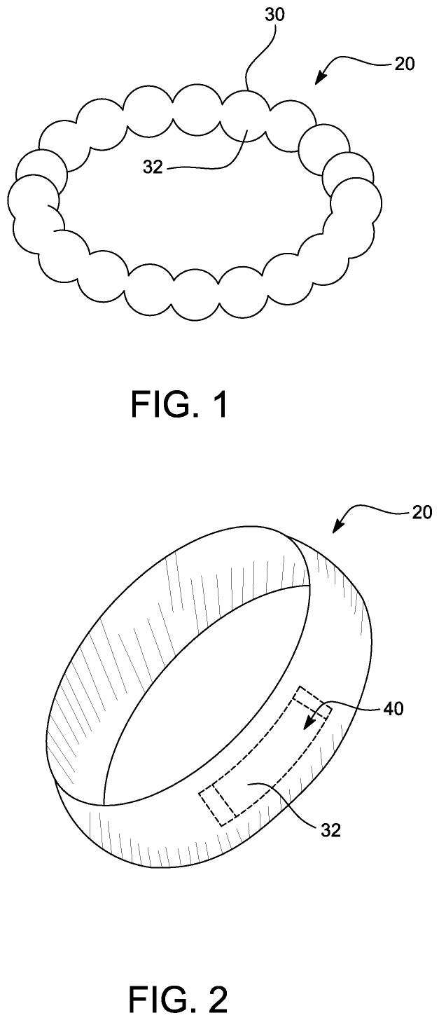

FIG. 1 schematically illustrates a disposable medicinal wristlet 20 that is medicated and to relieve tension associated with joint pain, carpel tunnel, arthritis, and other discomforts pertaining to a wearer's wrist.

The wristlet 20 may be manufactured of a stretchable, flexible and comfortable material such as an elastomeric material. In one embodiment, the wristlet 20 contains a medication 32 within, for example, a multiple of nodules 30. The medication 32 may include that which provides pain relief, and/or includes aromatherapy oils, natural essential oils or other such medications. That is, the term “medication” as utilized herein includes that which requires a prescription, that which does not include a prescription medication as well as aromatherapy oils, natural essential oils and/or other elixirs. Some example medications include treatments such as BLUE ICE GEL™ which is a temporary pain relief with menthol, ICY HOT™ which is first cold to dull the pain then hot to relax it away, and VICKS VAPOR RUB™ which temporarily relieves minor aches and pains of muscle and joints.

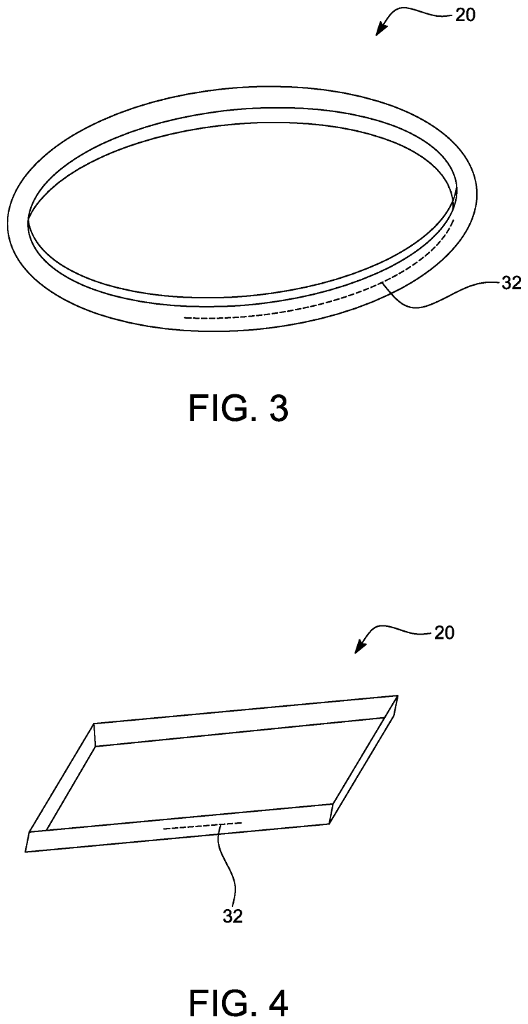

Although the wristlet 20 may include separate nodules 30, alternative embodiments may include other enclosed spaces 40 that contain the medication 32 (FIG. 2). In still other embodiments, the elastomeric material may be manufactured with the mediation 32 integral thereto. That is, no separate enclosed spaces or nodules are required as the medication 32 is formed as a component part of the jelly like elastomeric material (FIG. 3). In still other embodiments, the wristlet 20 may be of other shapes such as a rectilinear shape (FIG. 4)

The wristlet 20 may be manufactured in various sizes such that the wristlet 20 forms a contiguous ring or band that is tight on the wearer's wrist. The wristlet 20 is of sufficient flexibility to fit over the wearer's hand.

Each wristlet 20 may used as a set such that, for example a five (5) pack of wristlets may be used in series to promote mental health, balance the mind, body and soul, e.g., stress relief, energy, sleep, calmness, and balance. Each wristlet in the set may be infused with specific aromatherapy and natural as the wristlet may promote mental health as well as physical health. The set may be color coded and may include pastel colors, e.g., stress relief a light green, energy a light orange, sleep is light blue, calmness is beige and balance is lavender.

The wristlet 20 are disposable and are not refillable. The wristlet 20 itself is the medication. There need not be a mechanism to dispense the medication which results in the wristlet being disposable after a predetermined time period such as 8 hours. The medication 32 combined with natural body heat and pressure from the tightness of the wristlet 20 is operable to relieve wrist joint pain. That is, the medication 32 is administered in a transdermal manner in which application of the medication 32 is through the skin so that it is absorbed slowly into the body.

The wristlet 20 is discreet, comfortable, stylish and may be offered in neutral and colorful options. The wristlet 20 can be worn while playing sports and daily tasks.

Although the different non-limiting embodiments have specific illustrated components, the embodiments of this invention are not limited to those particular combinations. It is possible to use some of the components or features from any of the non-limiting embodiments in combination with features or components from any of the other non-limiting embodiments.

The foregoing description is exemplary rather than defined by the limitations within. Various non-limiting embodiments are disclosed herein, however, one of ordinary skill in the art would recognize that various modifications and variations in light of the above teachings will fall within the scope of the appended claims. It is therefore to be appreciated that within the scope of the appended claims, the disclosure may be practiced other than as specifically described. For that reason the appended claims should be studied to determine true scope and content.

What is claimed is:

1. A disposable medicinal wristlet, comprising:
a material formed as a contiguous band; and
a medication within the material, the medication is operable to be administered in a transdermal manner.

2. The wristlet as recited in claim 1, wherein the medication is integral with the material.

3. The wristlet as recited in claim 1, wherein the medication is contained within the material.

4. The wristlet as recited in claim 1, wherein the medication is contained within a enclosed space in the material.

5. The wristlet as recited in claim 1, wherein the medication is contained within a nodule formed by the material.

6. The wristlet as recited in claim 1, wherein the material is an elastomeric material.

7. A method of treating discomfort pertaining to a wearer's wrist, comprising:
wearing a wristlet operable to dispense a medication administered in a transdermal manner.

8. The method as recited in claim 7, wherein the wristlet is a contiguous elastomeric material band.

9. The method as recited in claim 7, wherein wearing the wristlet comprises exerting pressure on the wearer's wrist.

10. The method as recited in claim 7, wherein the medication is administered from nodules that contain the medication.

11. The method as recited in claim 7, wherein the medication is administered from an enclosed space that contain the medication.

12. The method as recited in claim 7, wherein the medication is administered from an elastomeric material that contain the medication.

13. The method as recited in claim 7, wherein the wristlet is one of a set.

14. The method as recited in claim 13, wherein each wristlet in the set is worn in a specific order.

15. The method as recited in claim 14, wherein each wristlet in the set is color coded.
